(19) United States
(12) Patent Application Publication
(10) Pub. No.: US 2007/0015230 A1
(43) Pub. Date: Jan. 18, 2007

(54) Identification and characterization of analytes from whole blood

(76) Inventors: David J. Hammond, Laytonsville, MD (US); Julia Tait Lathrop, Falls Church, VA (US)

Correspondence Address: MERCHANT & GOULD PC, P.O. BOX 2903, MINNEAPOLIS, MN, 55402-0903, US

(21) Appl. No.: 11/454,801

(22) Filed: Jun. 19, 2006

Related U.S. Application Data
(63) Continuation-in-part of application No. 10/414,523, filed on Apr. 14, 2003.
(63) Continuation-in-part of application No. 10/601,032, filed on Jun. 20, 2003.
(60) Provisional application No. 60/372,091, filed on Apr. 15, 2002.
(60) Provisional application No. 60/395,038, filed on Jul. 11, 2002.

(51) Int. Cl. C12Q 1/60; C12Q 1/37

(52) U.S. Cl. 435/11; 435/23

(57) ABSTRACT

The invention provides a method of identifying and detecting targets in blood samples by binding to multiple ligands. The method comprises providing ligands attached to a support, and contacting the ligands with targets in blood to allow at least one target to bind to at least one ligand. The method further comprises removal of non-bound targets and cellular components such as blood cells, platelets, abundant plasma proteins followed by dissociation and elution of the target(s). The eluted targets are detected by a variety of means in concentrations which are a function of their presence in one sample as compared with their concentration in a second sample and are simultaneously enriched for trace components.

### CROSS REFERENCE TO RELATED CASES

This application is a continuation-in-part of U.S. patent application Ser. No. 10/414,523, filed Apr. 14, 2003, which claims priority to U.S. Provisional Patent Application Ser. No. 60/372,091, filed Apr. 15, 2002, the contents of each of which are incorporated by reference herein in their entirety. This application is also a continuation-in-part of U.S. patent application Ser. No. 10/601,032, filed Jun. 20, 2003, which claims priority to U.S. Provisional Patent Application Ser. No. 60/395,038, filed Jul. 11, 2002, the contents of each of which are also incorporated by reference herein in their entirety.

### BACKGROUND

1. Technical Field

The field relates to sample preparation devices for the improved detection and characterization of analytes at concentrations that are a function of their original starting concentration for improved diagnostics and biomarker discovery from plasma proteins present in whole blood without the need for pre-fractionation.

2. Background of the Technology

Proteomics seeks to identify and characterize multiple proteins simultaneously. Blood is the primary useful specimen for analysis of existing and new biomarkers and diagnostics; however, it also comprises the largest and deepest version of the human proteome spanning 10¹⁰ or more orders of magnitude of concentration. Moreover, the number of proteins present is immense, particularly when considering post-translational micro-heterogeneity, variations in glycosylation, proteolytic fragmentation, protein-protein complexes and the antibody repertoire which, itself, may comprise 10,000,000 different proteins.

The enormous depth in concentration and complexity of blood reflects the dynamic range (difference between the highest and lowest concentration) (Lathrop, J. T., Carrick, K., Hayes, T. K., Hammond, D. J. (2005) “Rarity Holds a Charm”, Evaluation of trace proteins in plasma and serum (invited review), Expert Review of Proteomics 2 (3): 393-406; and Anderson (J. Physiol 563(1):23-60 (2005), Topical review Candidate-based proteomics in the search for biomarkers of cardiovascular disease), Anderson N. L. and Anderson, N. G, (“The human plasma proteome, History, Character and diagnostic prospects,” Molecular and Cellular Proteomics 1(11): 845-867 (2002)) with hemoglobin at >100 mg/ml, albumin at about 40 mg/ml and cytokines such as IL-6 at ˜1-10 pg/ml or lower. Current technology, i.e. mass spectrometry coupled with liquid chromatography and 2-dimensional gel electrophoresis coupled to mass spectrometry is limited to a dynamic range of 10³-10⁴ orders of magnitude relative to the abundant proteins.

Within whole blood there exists several proteomes, e.g. red blood cells, monocytes, lymphocytes, granulocytes, macrophages, platelets and the soluble plasma proteins. The plasma proteome is considered to be the most valuable since it contains, in addition to plasma proteins, leakage proteins and microparticles from damaged cells that may be important indicators (biomarkers) of disease. Preparation of plasma requires centrifugation of whole blood or plasmapheresis. Serum is also used as a source of plasma proteins. Serum is produced from plasma when blood is taken in the absence of anticoagulants. The proteins of the coagulation cascade, many of which are proteases, are activated resulting in the proteolytic digestion of fibrinogen to fibrin and the production of a fibrin clot. Many proteins and cells are trapped within the clot while the activation of proteases may degrade other soluble proteins. Thus, these processes for plasma protein collection can activate proteases and generate artifacts unrelated to unprocessed whole blood. Because of the above limitations, it is preferred to minimize the time and manipulation of the blood sample and to avoid fragmenting significant numbers of cells especially red blood cells which results in the liberation of high levels of hemoglobin into the plasma. Similarly, it is important to prevent the activation of platelets that produce a wide variety of cytokines and growth factors. Furthermore, the processing must not activate complement or coagulation factors, all of which may result in significant changes in the state and composition of plasma proteins. In addition, it is desirable to concentrate the trace components, decrease the abundant plasma components yet maintain concentration differentials in individual analytes between whole blood samples.

There are a number of approaches for selective enrichment of trace entities particularly plasma-derived proteins, but these are not generally directly applicable to use with whole blood. Proteins in plasma or serum may be digested by proteases, especially trypsin, and the resulting peptides may then be subjected to fractionation by multi-dimensional chromatography prior to analysis by mass spectrometry or tandem mass spectrometry, i.e. “MudPIT”. Alternatively, the plasma proteins themselves may be pre-fractionated by chromatography, e.g. ion exchange, reverse phase, metal chelate, gel filtration, and protein-specific or group-specific affinity separation prior to analysis. See Lee, W-C and Lee K H, “Applications of affinity chromatography in proteomics,” Analytical Biochemistry 324:1-10 (2004), for a review on affinity depletion, metal chelate affinity for enrichment of group specific proteins (see Shi, Y., Xiang R., Horvath C., and Wilkins J A “The role of liquid chromatography in proteomics,” J. Chromatography A 1053:27-36 (2004) for a more general review general on other forms of chromatography in proteomics).

One approach for improving the detection of trace components is to deplete selectively the abundant proteins in plasma or serum by use of specific monoclonal antibody affinity columns. Selective depletion strategies are most often targeted to albumin, IgG, IgA, transferrin, haptoglobin, alpha-1 proteinase inhibitor (API, also called alpha-1 antitrypsin), and fibrinogen. These monoclonal affinity columns are expensive, and seldom totally specific for their target. Furthermore, they only decrease the concentration range by a couple of orders of magnitude, leaving the trace proteins still below the limits of detection and still masked by the next set of most abundant proteins.

Thus, the above strategies will fractionate major abundant species from trace components in plasma or serum, but all have significant disadvantages including making the sample unstable because of the absence of the abundant proteins and further diluting trace components during processing. Moreover, during fractionation, many proteins that bind to the abundant species, especially albumin, antibodies, fibrinogen and alpha-2 macroglobulin are also depleted. Furthermore, these methodologies, especially monoclonal antibody-based depletion, are specific only for the proteins in an individual tissue from a single, or closely related, species. Finally, the immunoglobulins which are the most valuable class of proteins as biomarkers of infection and as therapeutics are frequently removed and generally are not available for evaluation.

Our priority application, Hammond and Lathrop (U.S. patent application Ser. No. 10/414,523, “Method for detecting ligands and proteins in a mixture”) teaches a technology now termed the “Bead Blot” that uses combinatorial libraries to bind representative amounts of most, if not all, the proteins present in a sample on an inert support and to transfer these proteins under one or more different conditions to a membrane. Since all the components within a sample can be captured in unique positions on a second matrix, the second matrix can be screened sequentially or simultaneously for the presence of multiple, independent targets. This technology was designed, in part, to identify proteins present in a complex mixture such as whole blood and those associated with a diseased state. Importantly, the Bead Blot can be used to quantify the amount of any one target on the membrane.

Alternatively the combinatorial library beads (ligand-support complexes) with the representative amounts of bound targets instead of being placed in a matrix and the proteins eluted can be subdivided and evaluated for a desired chemical composition (e.g. mass spectroscopy or gel electrophoresis), biochemical (e.g. enzyme activity or binding interaction), or biological activity (e.g. cell growth, death or differentiation). Examples of a chemical activity are a mass spectrum, or chemical composition. In Hammond D J, Lathrop J T, Sarkar J, Gheorghiu, L. WO 2004/007757, the desired activity optionally can be directly traced back to the individual bead, or sub-pool of beads from which it was selectively bound.

Boschetti and Hammond (“Methods for reducing the range in concentrations of analyte species in a sample” U.S. patent application Ser. No. 11/089,128) employ combinatorial technology for sample preparation which relates specifically to the compression of the analyte concentration range by decreasing the variance of a number of different analytes. This was achieved using the combinatorial libraries described by Hammond and Lathrop (in the priority application Provisional application No. 60/372,091, filed on Apr. 15 2002 and U.S. patent application Ser. No. 10/414,523) as the binding moieties and evaluating the bound and then eluted targets from plasma en masse or in sub-pools as described in (WO2004/007757), or through following elution by different sequential or parallel elution conditions as described in the “Bead Blot” (U.S. Provisional Application No. 60/414,523).

Boschetti and Hammond state that “at one extreme, the relative amount of binding moieties to analytes may be so large that the binding moieties are able to capture all of the analytes in the sample. In this case, there is no compression of the analyte concentration range. At the other extreme, the relative amount of binding moieties to analytes may be so small, that every analyte species saturates the ability of the binding moieties to bind. In this case, theoretically, the amount of each analyte species captured is the same, and the range in analyte concentration is compressed to equality. This extreme is particularly useful when the goal is to detect as many species as possible. Between these two extremes is the situation in which the more abundant species saturate the binding moieties, while the less abundant species do not saturate the binding moieties. In this case, there is little difference in concentration of the less abundant species that remain.”

A major problem with the conceptual approach of “Equalization” is the necessity to operate within a specific concentration range of analytes to ligands. Moreover, in biomarker discovery it is important to measure the relative concentration as well as the presence of an analyte. For example, patients with a steady state blood CRP level above 3 mg/L are considered to be at risk while those with higher, but decreasing levels may be recovering from a natural acute phase reaction. Those at levels below 1 mg/L are considered to be normal. In addition, increasing levels of a biomarker over time may indicate ongoing and developing tissue damage, e.g. in myocardial infarction while static, but elevated levels may indicate chronic disease. In addition, the discovery of new biomarkers will not be restricted to a single concentration range of analytes, thus it is necessary to preserve the relative concentrations of more abundant analytes directly from blood.

Consequently, to identify novel trace components present in whole blood it is necessary to invent a new, rapid, and easy to use technology that selectively enriches trace plasma components over many orders of magnitude, directly from whole blood. This inventive method addresses the above limitations and can be used easily and directly with whole blood without prior depletion of blood cells, including red blood cells, white cells and platelets, or the abundant blood plasma proteins, and can be used for quantifying the amount of target in a blood sample associated with a disease versus a normal state.

### SUMMARY

This invention provides significant improvement to the preparation of plasma targets especially trace proteins and pathogens for proteomic analysis directly from whole blood without the need for pre-fractionation. It incorporates compression of a range of protein concentrations between highly abundant species and maintains proportional amounts of any given analyte in one sample relative to the amount in a second comparable sample of blood.

The ability to detect relative amounts of a given analyte depends upon the matrix in which it is originally present. Frequently, it is desired to identify and quantify plasma proteins for proteome research and diagnostics which are present in whole blood; however, the complexity of whole blood and the dynamic concentration range of proteins make it impossible to identify more than about 10⁴ of the anticipated dynamic range of >10¹⁰ orders of magnitude. In addition, the huge complexity of the number of different proteins present and their post-translational modifications present enormous analytical limitations.

This invention provides one thousand or more ligand-support complexes that are designed to bind to plasma proteins in whole blood and to capture the plasma proteins in a manner that reflects their starting concentrations. This is achieved by providing ligands on a matrix that is chemically, biochemically and biologically inert.

Briefly the sample, e.g. anticoagulated whole blood containing red blood cells, white cells, platelets and plasma proteins is mixed with the ligand-support complexes, optionally with a dialysis compartment for targeted sequestering of selected proteins. The (plasma) proteins are allowed to bind to the beads and non-bound cells and other entities are removed by washing. The bound proteins are then eluted, detected and analyzed by a variety of physical, chemical, biochemical or biological methods.

### BRIEF DESCRIPTION OF THE DRAWINGS

FIG. 1 Binding and elution of proteins in whole blood treated with and without PPACK, CPD (commercial citrate anticoagulant), EDTA, citrate, and heparin. Lane 1: MW standard: Lane 2: serum; Lane 3: WB/CPD/with PPACK; Lane 4: WB/CPD/without PPACK-; Lane 5: WB/EDTA/with PPACK; Lane 6: WB/EDTA/without PPACK; Lane 7: WB/Citrate/with PPACK; Lane 8: WB/CITRATE/without PPACK; Lane 9: WB/Heparin/with PPACK; and Lane 10: WB/Heparin/without PPACK.

FIG. 2 Comparison of blood vs plasma binding. Lane 1: whole blood before binding to ligands, Lane 2: proteins in whole blood not bound to library (flow through, or FT); Lane 3: plasma fraction of the whole blood in lane 2; Lane 4: proteins from whole blood that bound to ligands; Lane 5: proteins from plasma that bound to ligands; Lane 6: proteins from plasma not bound to the library (FT); Lane 7: plasma before binding to ligands; and Lane 8: molecular weight standard.

FIG. 3 Binding of prion protein (PrPres) spiked into whole blood. Lane 1: molecular weight marker; Lane 2: IgG “medium” standard; Lane 3: IgG “high” standard; Lane 4: 0.1% Scrapie infected brain homogenate (SBH) in buffer no proteinase K (PK) digestion; Lane 5: 0.1% SBH in buffer +PK; Lane 6: ligand 1/buffer −PK; Lane 7: ligand 1/buffer +PK; Lane 8: ligand 1/incubated WB −PK; Lane 9:ligand 1/incubated whole blood +PK; Lane 10: ligand 1/not incubated whole blood −PK; Lane 11: ligand 1/not incubated whole blood +PK; Lane 12: ligand 2/buffer −PK; Lane 13: ligand 2/buffer +PK; Lane 14: ligand 2/incubated whole blood −PK; Lane 15: ligand 2/incubated whole blood +PK; Lane 16: ligand 2/not incubated whole blood −PK; and Lane 17: ligand 2/not incubated whole blood +PK.

FIG. 4. Detection of Troponin by Western Blot using spiked whole blood samples treated and untreated with the combinatorial ligand library . . . .

FIG. 5. Detection of Troponin by ELISA in whole blood and combinatorial ligand library-treated whole blood samples.

FIG. 6. Detection of Troponin in plasma spiked with Troponin, blood spiked with Troponin then treated with the library, and blood treated with the library then spiked with Troponin, as detected by Bio-Quant ELISA kit.

### DETAILED DESCRIPTION

The invention provides a methodology to detect simultaneously target molecules, following separation from a sample of blood in an amount which is a function of its concentration in blood plasma. In particular, the invention provides a simple to use technology of detecting and analyzing a target in blood that binds to a ligand. The method comprises (i) providing one thousand or more ligands, wherein each ligand is attached to a support to form one thousand or more ligand-support complexes, (ii) contacting the ligand-support complexes with a blood sample under conditions that allow at least one target to bind to at least one ligand-support complex, thereby forming one or more target-ligand support complexes, (iii) removing the non-bound, cell-associated components, abundant plasma proteins, etc., (iv) eluting at least a portion of the target of at least one target-ligand-support complex in an amount proportional to its presence in the starting sample and (v) detecting the target, and optionally analyzing the relative amount of the target that binds to one or more ligands.

Although the preferred embodiment uses one thousand or more different ligands to produce one thousand or more ligand-support complexes, one of skill in the art could envision the use of fewer ligands. The amount of ligands used, or course, will depend on the complexity of the sample being characterized. Although most samples to be characterized are complex enough to require one thousand or more ligand-support complexes, some may only require 900, 800, 700, 600, 500, 400, 300, 200, 100 or fewer. Furthermore, as many as five thousand, ten thousand, fifty thousand, one hundred thousand, five hundred thousand, one million or more different ligand-support complexes can be used in the methods of the claimed invention.

Alternatively, the method optionally comprises dividing two or more target-ligands-support complexes before step (iv) into sub-pools as described in the parent application, U.S. patent application Ser. No. 10/601,032, then eluting at least a portion of a target of at least two target-ligand-support complexes from one sub-pool in an amount proportional to the amount captured from the starting sample, which is related to its original concentration in the starting sample and detecting and analyzing the at least one target and (v) detecting the target whereupon the relative amount of the target that binds to two or more ligands is characterized.

The method also optionally comprises conducting step (iv) in a medium containing a competitive binding agent, which binds to the target of at least one target-ligand-support complex, thereby causing the ligand to dissociate from at least a portion of the target. These competitive binding agents can be a ligand (different from the ligand of the target-ligand-support complex), cofactors for the target, enantiomeric specific molecules, and the like.

The invention offers a number of advantages over previous target detection and analysis methods using blood plasma-derived targets. Currently, blood samples must be centrifuged to obtain plasma proteins for analysis. This is time consuming, expensive and often impractical for point of care diagnosis of disease. Furthermore, the length of processing time allows for activation of plasma proteases, and can generate proteolytic fragments that may vary between samples, but are unrelated to the physiological state of the sample when obtained. Alternatively, serum formation from blood is frequently used to prepare samples for analysis. This procedure involves activation of a number of proteins of the coagulation cascade to form a clot and separation of the clot which primarily contains fibrin, other plasma proteins, red blood cells, and platelets from the non-clotted “serum” proteins. During clot formation analytes in plasma may be degraded by the activated coagulation enzymes and may also be sequestered into the clot, excluding them from subsequent analysis. Serum and whole blood frequently have high levels of hemoglobin as a result of red blood cell lysis, possibly due to the collection conditions; high levels of hemoglobin interfere with many analytical assays. The method of the invention recovers plasma proteins from whole blood without the need for pre-fractionation thereby saving time, manipulation and capturing the targets as close to their physiological state as possible.

In a preferred embodiment, the test sample is whole blood and the targets are plasma-derived proteins. Whole blood requires the presence of anticoagulants to prevent coagulation over time. Preferred anticoagulants include ethylene diaminetetra acetic acid (EDTA), citrate, heparin, and protease inhibitors such as aprotinin and D-phenylalanyl-L-prolyl-L-arginine chloromethylketone (PPACK). Protease inhibitors should be included when heparin is used as the anticoagulant as heparin also binds to ligands on the supports. The preferred contact time is kept as short as possible to prevent undue protein modification over time. A contact time of ≦15 mins is preferred with other times ranging up to 24 hours and beyond. The preferred ratio of combinatorial library to whole blood may be in the order of 1:1 to 1:10 to 1:100 to 1:1,000 or more.

A further advantage of the targets being immobilized as target-ligand-support complexes is that binding helps stabilize the structure of some targets (see U.S. Pat. No. 5,786,458 to Baumbach, Hammond, Lang and Galloway, for rationale, examples and references). Although this was used by Baumbach et al for improved and specific viral inactivation of therapeutic proteins, the same general principle holds for the multiple targets bound to multiple ligands of this invention. Thus, the inventive method will simultaneously bind and concentrate trace plasma proteins on their respective ligands from whole blood without the need for generation of plasma through centrifugation or serum collection, and it will eliminate the majority of abundant species including whole cells, abundant proteins such as hemoglobin, albumin and transferrin that are present in levels far greater than the capacity of the library, while immobilizing and stabilizing the bound analytes on the ligands. This immobilization will physically restrict the ability for proteins to freely diffuse into solution and interact and potentially degrade important biomarkers on other target-ligand-support complexes. In addition, since the immobilization of proteins will stabilize some target's conformational structure as target-ligand-support-complexes the ability to evaluate biochemical and biological properties of the bound targets is potentially improved.

Conventionally, the resulting plasma or serum is further fractionated to improve the sensitivity of detection of the trace proteins. This can be achieved by increasing the relative proportion of the trace components by selectively depleting the abundant proteins using monoclonal affinity columns directed against the most abundant proteins, fractionation using chromatography chips or columns (ion exchange, hydrophobic, metal chelate, gel filtration) or may be achieved post-digestion of the proteins by trypsin and separation of the ensuing peptides using 2-D LC followed by tandem mass spectroscopy. Frequently, the methods of choice for 2-D LC are ion exchange and reverse phase. All of these strategies have significant advantages and disadvantages. The greatest disadvantages are time, cost, losses and dilution of trace targets during manipulation of the plasma or serum sample and sample instability at low analyte, especially protein (particularly albumin) concentrations.

This inventive method overcomes many of these disadvantages by concentrating trace plasma targets in amounts that are a function of its concentration in the original blood sample when similar blood samples are compared. Moreover, in general, trace plasma targets are preferentially concentrated relative to more abundant species making the detection of proteins preferentially expressed in a disease state at low levels easier to identify. In addition, because all the components within a sample can be captured on different beads (ligand-support-complexes) the beads may be assayed in total sequentially or simultaneously for the presence of multiple, independent targets, or may be split into a number of different sub-pools, or the individual beads may be evaluated independently for the presence of targets.

Yet another advantage of the invention is that the biological, biochemical, and chemical activity of the target can be maintained, if desired, by carefully selecting the elution conditions. Elution conditions can be advantageously controlled to recover a subpopulation of the bound target at any one time, and to identify specific elution conditions of selected targets. Moreover, it is also possible to identify targets that bind to specific molecules by using an elution buffer containing that specified molecule or conjugate of that molecule (U.S. patent application Ser. No. 10/414,523).

A further option of the method of this invention is to concentrate the plasma proteins on the beads while decreasing the concentration of the most efficiently bound proteins such as fibrinogen and lipoproteins such as LDL and HDL. The capture of the highly interactive proteins may be performed prior to and/or during the capture of the plasma proteins on the non-targeted ligand support complexes. The amount of any one analyte bound to a combinatorial library varies significantly. For example, fibrinogen and HDL have a high incidence of high affinity ligands perhaps due to such features as stronger and more effective binding through multi-point attachment of the ligands on individual beads to identical subunits on the target. In addition, different subunits may bind to different ligands increasing the number of possible interactions while each subunit may have multiple binding sites. Furthermore, proteins such as fibrinogen exist in protein complexes and binding of the complex may be mediated by binding any one member of the complex to the support. Fibrinogen itself is comprised of six subunits and binds many other proteins, including fibronectin, factor XIII, von Willebrand factor to name, but a few. The HDL complex contains paraoxonase, apolipoprotein (apo) A1, apo All, apo IV, apo B100, apo D, apo E and other proteins. In contrast, transferrin and A1PI that have a low frequency of high affinity ligands circulate in the blood as monomers with very few binding interactions with other proteins and binding sites. An example of this approach is provided in the parent application (U.S. patent application Ser. No. 10/414,523) for whole plasma, but is similarly applicable to blood.

Affinity resins specific to proteins that preferentially bind to a library, (e.g. fibrinogen, immunoglobulin, HDL, and LDL, etc.) may be included in a compartment separated from the library by a dialysis membrane to further enrich for binding of the trace targets. The advantages of performing this approach instead of conventional depletion strategies is that entities binding to the major species can still be captured on the library beads (ligand-support-complexes) without being almost completely lost to the bound abundant species. In addition, a proportion of the abundant proteins will remain in solution to bind to high affinity binding sites within the library and will still be analyzed albeit at a lower overall concentration. Several high affinity resins specific for different antibodies or targets may be included in the same compartment. A convenient method for finding ligands is taught in the parent application (U.S. patent application Ser. No. 10/414,523). The invention teaches that the composition of plasma targets within blood may be rapidly analyzed with minimal manipulation over a very broad range of target concentrations and the amount of bound protein is a function of the amount of free plasma derived target in the starting sample. Moreover, the degree of competition for binding may be modulated by reduction in the free concentration of targets that bind with high frequency to the ligands.

Ligands

For purposes of the invention, the term “ligand” as used herein refers to any biological, chemical, or biochemical entity, such as a compound that binds to a target. It is important to note that two or more targets can compete for binding to one or more ligands. The ligand can be isolated from natural or synthetically produced materials. Suitable ligands for the inventive method include, but are not limited to, amino acids, peptides, antibody preparations (e.g. antibody fragments, chemically modified antibodies, and the like), carbohydrates, sugars, lipids, organic molecules, and combinations thereof.

Organic molecules include, for example, synthetic organic compounds typically employed as pharmacotherapeutic agents. Such molecules are, optionally, mass produced by combinatorial methods or, more specifically, by strategic syntheses devised to arrive at specific molecules. Likewise, organic molecules also include natural products and analogues, whether extracted from their natural environment or strategically synthesized. Organic molecules include amino acids, peptides, nucleic acids, carbohydrates, sugars, lipids, steroids, drugs, vitamins, cofactors, etc. The term “organic” as used herein is not intended to be limited to molecules comprised only of carbon and hydrogen, but rather is used in its broader sense encompassing macromolecules of biological origin.

Preferably the ligands are peptides. More preferably, the peptides consist of essentially of about 2-15 amino acids. The term peptide as used herein refers to an entity comprising at least one peptide bond, and can comprise D and/or L amino acids. Ideally the ligand is between 3 and 7 amino acids. If desired the peptide can be generated by techniques commonly employed in the generation of combinatorial peptide libraries, e.g. the split, couple, recombine method or other approaches known in the art (Furka et al., Int. J. Peptide Protein Res., 37:487-493 (1991); K. S. Lam et al., Nature, 354:82-84 (1991); WO 92/00091 (1992); U.S. Pat. No. 5,133,866; U.S. Pat. No. 5,010,175; U.S. Pat. No. 5,498,538; expression of peptide libraries is described by Devlin et al., Science, 249:404-406 (1990)). In peptide libraries, the number of discrete peptides of different sequences increases dramatically with the number of cycles of coupling reactions performed and the number of separate reactions per cycle. For example, the random incorporation of 19 amino acids into pentapeptides produces up to 2,476,099 (19⁵) individual peptides of differing sequence (Lam et al., Nature, 354:82-84 (1991)). Combinatorial methods allow synthesis of libraries of ligands directly on a support. Typically the ligands are synthesized on particles of support media in such that multiple copies of a single ligand are synthesized on each particle (e.g. bead), although this is not required in the context of the invention. The preferred density of the ligands is 50-400 μmol/gram dry weight, preferably 50 to 150 μmole/gram dry weight, and most preferably 100 μlmole/gram dry weight.

Examples of amino acids that may be included in the library are the natural occurring L-amino acids and their D-isomers. Other amino acids that may be included in the ligands of the library are: aminodipic acid, beta alanine, 2-aminobutyric acid, 6-amino caproic acid, citrulline, hydroxylysine, N-methylvaline, and norleucine to name, but a few. The amino acids may be modified prior to incorporation into the library, e.g. through phosphorylation of serine, threonine and tyrosine. In addition, the ligands may be modified post-synthesis by chemical or biochemical means. Examples of chemical modification include acetylation of amino groups with acetic anhydride, reaction with aziridines, epoxides, and methylglyoxal. Some modifications are the result of Maillard reactions and such products in tissue proteins are implicated in the pathology in aging, e.g. advanced glycation end-products and glyoxidation products such as (carboxyethyl)lysine. Other modifications may be enzymatic through the action of protein kinases that phosphorylate serine, threonine and tyrosine, and glycosylases that glycosylate asparagine, serine and threonine.

Combinatorial libraries of ligands are combinations of any of the above listed ligands. For example, a combinatorial library of ligands can consist of mixtures of peptide hexamers. Alternatively, the combinatorial library of ligands can consist of mixtures of all lengths of peptides. The combinatorial library of ligands can also consist of a mixture of peptides, amino acids, proteins, antibodies, etc.

Ligand-support complexes specific to abundant species have been identified by screening combinatorial libraries as described in U.S. patent application Ser. No. 10/414,523 and U.S. patent application Ser. No. 10/601,032. Alternatively they may be obtained from hybridoma technology and from commercial sources. Such commercial suppliers include Agilent Technologies' multi affinity removal system, Genway Biotech's “Seppro” and Sigma-Aldrich's ProteoPrep 20 immunodepletion Kit.

Target

For purposes of the present invention, the term “target” as used herein refers to any chemical, biochemical or biological entity, such as a molecule, compound, protein, virus, microparticle, or organelle, that is present in blood and binds to a ligand. For example, the target can be a drug or drug candidate (such as a small molecule drug candidate), a toxin, an epitope specific antibody or an infectious agent. In addition the target can be a bacterium, a fungus, a yeast or a parasite. Suitable targets for the inventive method include but are not limited to cells, viruses, bacteria, yeast, microparticles, proteins, protein complexes, peptides, amino acids, nucleic acids, tissue leakage proteins, carbohydrates, lipids, drugs, synthetic inorganic compounds, synthetic organic compounds, isoforms of any of the foregoing, and combinations of any of the foregoing. By isoforms it is intended to mean proteins, protein complexes, peptides, and nucleic acids that differ from the native protein, protein complex, peptide or nucleic acid. Such a difference can be structural, in which the primary amino acid sequence is the same but the three-dimensional structure differs. Preferably, the targets are proteins. Suitable proteins targets include, for example, receptors, antibodies, immunogens, enzymes (e.g. proteases), detoxification proteins, acute phase reactants and proteins involved in inflammation, e.g. C-reactive protein. More preferably, the proteins are found as the product of cellular breakdown, e.g. microparticles and other biomarkers of disease, e.g. c-reactive protein, troponin, and infectious agents such as prion. Such proteins in blood include, for example, normal prion protein, proteases, epitope-specific antibodies, complement factors, fibrinogen, A1PI, or coagulation factors, all of which are naturally found in the blood of an organism in a non-diseased state. Alternatively, the blood protein is present in plasma associated with a diseased state (optionally not found in the plasma of a healthy subject) or as a result of the administration of an agent, e.g. a drug. In this regard, the plasma protein can be an infectious PrPsc prion protein.

One advantage of the inventive method is the ability to identify and/or characterize targets on the basis of chemical, biochemical and biological activity, without prior knowledge of the target's molecular identity. The chemical activity may be a mass spectral signal and the biochemical activity an enzyme activity such as a protease, organophosphatase an inflammatory cytokine, etc. Accordingly the target can display a biological activity and need not undergo processing prior to practicing the inventive method.

Supports

In one embodiment of the inventive method, the ligand is attached to a support. The term “support” as used herein refers to any support matrix, such as those solid supports known in the art, which serve to immobilize the ligand. Suitable supports include, but are not limited to, membranes, filters, meshes, beads or particles comprised of or coated with cellulose, acrylates, polyacrylates, polyhydroxymethacrylates, polystyrene, dextran, agarose, polysaccharides, hydrophilic vinyl polymers, polymerized derivatives of any of the foregoing and combinations of any of the foregoing, as well as any porous or non-porous matrix to which ligands can be synthesized. Preferably, the support is inert such that the chemical reaction with the target and/or ligand is minimized. Preferably the support is biochemically inert such that proteins activity, e.g. complement and coagulation proteins in blood are not activated by the support. Preferably the support is biologically inert such that cellular function is unaffected by the support. Most preferably the support can be used directly with whole blood without the need for prior fractionation to remove red and white cells, platelets, and lipids.

The criterion for selecting a base polymer for use as the support for synthesizing the ligand-support complexes is that it must be chemically, biochemically and biologically inert. Preferred supports are resin beads comprising a material selected from the group consisting of agarose, cellulose, dextran, ethylene glycol, fluoropolymers, polyacrylate, polyesters, polyethylene glycol, methacrylate and hydroxymethacrylates including glycidol methacrylate, ethylene glycol dimethacrylate, penta erythritol dimethacrylate, dimethacrylate, polyhydroxymethacrylate, polypropylene, polyethylene oxides, polysaccharide derivatives of any of the foregoing, and combinations of the foregoing. A particularly preferred support material is a polyhydroxylated methacrylate polymer. Examples of such resins include Toyopearl AF-Amino 650M from Tosoh Bioscience, fractogel EMD Amino (M) from MerckKGaA in Darmstadt, Germany, and Affi-Prep and MacroPrep media from Bio-Rad. Another preferred resin is a polymer of glycidol methacrylate, polyethylene oxide, penta erythritol and ethylene glycol dimethacrylate, or analogs and combinations thereof. The resin should also possess sufficient concentration of functionalized groups for the chemical synthesis of combinatorial libraries by the split, couple and recombine method of Furka as extended by Lam et al. (Furka et al., Int. J. Peptide Protein Res., 37:487-493 (1991); Lam et al., Nature, 354:82-84 (1991)). The base resin must not activate platelets, coagulation factors, or complement, must have low non-specific binding to cells, albumin and the other major proteins and must have no significant effect on the cell lines selected for evaluation in functional assays.

Many solid supports displaying potential ligands are commercially available. Alternatively, the one or more ligands of the inventive method can be indirectly attached or directly immobilized on the support using standard methods (Merrifield, R. B., J. Am. Chem. Soc. 85(14):2149-2154 (1963); Harlow and Lane, Antibodies, Cold Spring Harbor Laboratory, Cold Spring Harbor, N.Y. (1988); Biancala et al., Letters in Peptide Science, 7(291), 297(2000): MacBeath et al., Science, 289, 1760-1763 (2000); Cass et al., ed., Proceedings of the Thirteenth American Peptide Symposium. Leiden, Escom, 975-979 (1994); U.S. Pat. No. 5,576,220; Cook et al., Tetrahedron Letters; 35, 6777-6780 (1994); and Fodor et al., Science, 251(4995): 767-773 (1991)).

The preferred support is a macroporous resin bead which allows large molecular proteins to readily permeate the bead. A suitable pore size is 100 nm (1,000 Angstroms) allows most proteins and some viruses, but not cells to enter the pores. Larger porosity may be desired for selective targeting of viruses and microparticles.

The capacity of the bead for protein should be high to allow minimal volumes of beads to be used per unit volume of blood. This will limit the dilution of blood with large quantities of bead solvents and water of hydration. The average capacity of the resins used in this invention are about 10 mg/ml for whole blood though the surface capacity of the beads themselves are 30 sq. meters per gram dry weight of resin providing an optimal capacity of over 20 mg/ml.

In one embodiment the ligands are synthesized on the surface of a support, which is advantageous in generating peptide libraries. The ligands can be chemically conjugated to the support or can be attached via linkers, such as streptavidin, beta-alanine, glycine, methionine, polymers containing glycine and serine, (—O—CH₂—CH₂—)n where n is between 1 and 30, short chain hydrocarbons of the formula —(CH₂)—, polyethylene glycol, and epsilon amino caproic acid.

In the context of the inventive method, at least a portion of the targets of the target-ligand-support-complexes are dissociated from the ligand-support-complex. By eluting or dissociating at least a portion of the targets, it is meant that a percentage (or fragment) of any one specific target is eluted, since it is unlikely that 100% of the target bound to a specific ligand could be transferred. Thus, by “at least a portion” it is meant that at least 10%, 20%, 30%, 40%, 50%, 60%, 70%, 80% or 90% of the target of at least one target-ligand complex within the first matrix is transferred to the second matrix.

The dissociation of the target from the ligand is achieved through contacting the target ligand support complexes with a solution that promotes dissociation. The solution can be selected from buffers of known salt concentrations (2M NaCl), extremes of pH, or denaturing capability, e.g. strong chaotropes, e.g. 6M guanidine.HCl, organic solvents, de-ionized water. Alternatively, or in addition, an isoelectric gradient can dissociate the target from the ligand-support complex. Transfer solutions can also comprise ligands (different from the ligands on the ligand-support complexes), cofactors for the target, enantiomeric specific molecules, and the like. Use of different transfer solutions allow investigation of elution conditions and target sub-populations.

The dissociation conditions employed in the inventive method are selected to minimize disruption of the ligand from the support. In other words, the elution and transfer conditions should not release the ligand (or ligand-support complex) from the matrix (unless this is specifically desired).

Contact Conditions

The blood sample containing targets may be mixed with the ligand-support complexes in one of several different formats well known in the art for binding targets to a support (U.S. patent application Ser. Nos. 10/414,523 10/601,032). These formats include chromatography column formats, batch addition of ligand-support complexes, monolithic structures, membranes or arrays. The beads may be macroporous or may be milled to a fine powder. In addition, the beads may be made of a magnetic core. Alternatively the ligands to the most interactive proteins may be immobilized on dip-sticks.

The blood may be prepared in any one of the standard anticoagulants such as EDTA, citrate, heparin or in a protease inhibitor cocktail. Since heparin will bind to beads bearing ligands to heparin this anticoagulant is best used in the presence of additional protease inhibitor(s).

The contact temperature can be conveniently performed at ambient conditions at the point of blood collection 4-40° C. The incubation time may range from <5 mins to 24 hours and beyond and preferably from 15 mins to 60 mins. The resin may be removed by filtration, sedimentation under gravity or by differential centrifugation or the blood may be passed through the resin in a column format. In the case of dipsticks they may be conveniently removed by hand.

Beads of average diameter about 65 μm may be added to whole blood in a batch format. The mixture may be agitated at room temperature then filtered on a filter with sufficient porosity to let the red blood cells pass through (<10 μm). White blood cells including basophiles, neutrophiles, granulocytes (polymorphonuclear leucocytes), agranulocytes (mononuclear leucocytes), eosinophiles, B and T-cell lymphocytes are typically 10-16 μm in diameter and platelets are typically about 2 μm in diameter.

Large beads, greater or equal to about 200 μm in diameter may be used in chromatography format without clogging due to entrapment of red or white blood cells in the chromatography bed.

Reduction of Highly Interactive-Binding Proteins

Binding of the trace components from whole blood may be improved by binding of the trace components to a combinatorial library in one compartment while ligands that exhibit high affinity binding to the most interactive targets may be separated in another compartment. Equipment for performing this step (equilibrium affinity dialysis) is commercially available from companies such as Harvard Apparatus and SDR Molecular and may possess 2 or more chambers, and use semi-permeable membranes. In such a method of separating the most interactive targets from the remainder of the targets, target-specific affinity resins are used in one compartment and one thousand or more combinatorial ligand-support-complex are used in the other.

Other means of separating a portion of the most interactive targets from trace components include the use of magnetic beads or dip-sticks. Magnetic beads may be made by incorporating micronized magnetic particles into the synthesis of the resin or modifying the beads through reaction with magnetic particles. Such beads are available from BioScience Bead Division of CSS International and activated magnetic beads with terminal amines, epoxy-activated, hydrazide and aldehyde-modified beads are available from Bioclone Inc. The magnetic beads bearing ligands that bind to the most interactive species may be separated based on magnetic charge. Alternatively, the beads may be physically separated by sedimentation rate, density, and size. Alternatively, the beads may be fixed to supports such as dip-sticks, and membranes. Likewise, the specific affinity ligands can be included on the surface of dip-sticks or may be synthesized on, or coupled to the surface of a membrane. Separation may then be easily accomplished by physically removing the dipstick or membrane with the target-ligand-support complexes attached. In addition, proteins may be contacted with the selected resins prior to and during contact with the library.

Detection and Analysis of the Target

The inventive method further comprises detecting the dissociated plasma-derived targets that bind to the ligands of the ligand-support complexes. The term “detection” and words related thereto as used herein refer to the identification of any distinctive quality or trait of a target, and do not require that the precise chemical identities, e.g. the molecular formula, chemical structure, nucleotide sequence or amino acid sequence of the target is elucidated. Indeed, characterization of the targets may be performed individually, sequentially or simultaneously. Alternatively, the targets can be detected by testing for a property or activity of the target, such as biological property, chemical property, or a property that is a combination of any of the foregoing. The targets may be directly detected using, for example molecular weight by mass spectrometry or gel-electrophoresis, or spectral signal. Alternatively, the targets may be detected using immunological assays for example, ELISA, Western blot and nephelometry. Alternatively, the targets may be detected by means of an enzyme assay such as a protease or organophosphatase that hydrolyses a fluorogenic substrate to create a fluorescent signal. Alternatively the targets may be detected and analyzed by contacting cells with the eluted targets and detecting a cellular response using a biological assay such as cell growth, death, and differentiation. A further important property is the amplification of nucleic acid by polymerase chain reaction and other nucleic acid amplification techniques. Additional techniques for detection and analysis are reviewed in Phizicky E M and Fields S., Microbiol. Rev., 59(1):94-123 (1995).

The present invention also provides a method of identifying diagnostic biomarkers. In a preferred embodiment, the method comprises the steps of (a) providing a first blood sample from a first organism having a first phenotype and a first plurality of different targets, (b) providing a second blood sample with a second phenotype (preferably from a diseased individual) and a second plurality of different targets (c) performing the method for reducing the range in concentrations and preserving the proportional amount of targets on each of the samples, thereby creating a third and fourth sample set of biosamples, respectively; (d) detecting target species in each of the third and fourth set of biosamples, and identifying at least one target that is differentially present in the third and fourth sets of biosamples, whereby the at least one target species and its approximate concentration is a biomarker for distinguishing the first phenotype from the second phenotype.

### EXAMPLES

### Example 1

In order to be used with whole blood it is necessary that the support resin induce no activition of platelets, complement, or factor VII, nor cause hemolysis or other adverse effects on the red blood cells contained in whole blood. Activation of plasma proteins can lead to coagulation of the blood, while activation of platelets can lead to expression of cytokines that are not present in the original starting materials. These experiments were designed to evaluate the effect of the support resin on these critical whole blood parameters; however, plasma was used as the starting material in order to enrich for the tested proteins, thereby making the assay even more sensitive than if used with whole blood.

Methods:

Platelet-poor (for Factor VII and complement assays) or platelet rich plasma (for platelet activation assays) was applied to 0.5 ml columns of base resin with the flow rate controlled by peristaltic pump at 0.5 ml/min. Flow though was collected in aliquots and the level of platelet activation, complement activation, and factor VII activation was evaluated using commercially available kits and reagents:

Factor VII assay: measures the level of activated factor VII in a sample by measuring clotting time with factor VII-depleted plasma (by definition, 1 U/ml is the normal value for factor VII activity in plasma). Reagents: Reference plasma-Hemoliance cat # 49738740, Brain thromboplastin-Hemoliance cat # 49732400, FVII-deficient plasma-Hemoliance cat # 49738071, Owren's buffer-Hemoliance cat # 49738600. The assay is performed by reconstituting lyophilized materials in dH₂O and storing them on ice. A calibration curve was made with 1:10, 1:20, 1:40, and 1:80 dilutions of reference plasma in Owren's buffer. PPP sample was diluted 1:40 with Owren's buffer, and FVII-deficient plasma was diluted 1:5 with Owren's buffer. Coagulation was assayed in a Dade Behring BCS according to the manufacturer's instructions, by adding thrombospondin to the samples and reference plasma as directed.

Complement assay: performed according to the manufacturer's instructions in the kit, PROGEN Biotechnik, cat# PR 5901 using platelet poor plasma.

Platelet activation assay: comparing the ratio of the amount of CD-61, a housekeeping protein on the surface of all platelets as a measure of the number of platelets in a sample, to the amount of CD-62P, a protein that is exposed on the surface when platelets are activated as a measure of the number of activated platelets. The levels of activation are quantitated on a Becton Dickenson flow cytometer according to the manufacturer's instructions, to compare levels of IgG in the material as background. The following combinations were tested with the samples: IgG-FITC+IgG-PE, Anti-CD 61-FITC, IgG-PE, IgG-FITC+Anti-CD 62-PE, and Anti-CD 61-FITC+Anti-CD-62 PE. The samples tested with the above were PRP from flow through and PRP from flow through +PMA as a positive control. The levels of activation were compared with the standards.

TABLE 1
Assays used for plasma protein analysis

| Endpoint | Assay | Instrument |
|---|---|---|
| Platelet activation | CD61/CD62P (BD) | Becton-Dickenson flow |
| (%) | | cytometer |
| Complement activation | Complement C3a- | Molecular Devices |
| (ng/ml) | desArg ELISA | Plate reader |
| | (Progen) | |
| Factor VII activity | Coagulation | DB BCS coagulometer |
| (U/ml) | | |

Hemolysis of red blood cells was measured in several serial aliquots of flow through by assaying the amount of plasma hemoglobin using the HemoCue cuvettes, reagents, and instrument (HemoCue, Inc) and using the formula:

$$Hemolysis = \frac{\begin{matrix}[ 100 - sample hematocrit ( \% ) ] \times \\ sample supernatant hemoglobin ( mg \text{/} dL ) ]\end{matrix}}{sample total hemoglobin ( g \text{/} dL ) \times 1000}$$

Results:

The results for plasma protein and platelet activation are presented in Table 2. There was no activation of any of these parameters significantly above background; therefore, the base resin does not activate platelets, Factor VII, or complement.

The hemolysis results are presented in Table 3 which show that the base resin does not cause hemolysis of red blood cells.

TABLE 2
Plasma protein and platelet activation.

| | | | | LR Whole |
|---|---|---|---|---|
| | | Amino 650 M | | blood |
| | | volume of blood per 0.5 ml resin | | |
| Endpoint | Assay | 5 ml | 10 ml | NA |
| Platelet | CD61/CD62P | 0.105 | ND | 0.11 |
| activation (%) | | | | |
| Complement | Complement | 193 | ND | 147 |
| activation | C3a-desArg | | | |
| (ng/ml) | ELISA | | | |
| Factor VII | Coagulation | 0.61 | ND | 0.871 |
| activity (U/ml) | Coagulation | 0.77 | 1.33 | 1.08 |

ND = not done.

TABLE 3
Hemolysis of base resin

| | | Plasma HGB | Total HGB | |
|---|---|---|---|---|
| | HCT % | (mg/dL) | (g/dL) | Hemolysis % |
| challenge | 40 | 10 | 13.3 | 0.045 |
| FT 1 | 39 | 20 | 13.3 | 0.092 |
| FT 2 | 38 | 30 | 13.0 | 0.14 |
| FT 3 | 39 | 10 | 13.4 | 0.045 |
| FT 4 | 39 | 30 | 13.4 | 0.14 |
| FT 5 | 39 | 20 | 13.2 | 0.092 |

### Example 2

### Use of Different Anticoagulants

One of the chief advantages of this invention is the ability to use the library with whole blood, collected in various anticoagulants, without the addition of protease inhibitors. The speed at which blood is processed is a significant advantage, in that production of plasma and serum are known to produce artifacts. The purpose of this experiment was to determine if the addition of PPACK, an irreversible thrombin inhibitor, would alter the pattern of proteins detected following treatment of whole blood that was collected in different anticoagulants.

Methods:

Combinatorial peptide libraries of 6-mer peptides were synthesized on Toyopearl 650 AF-650 amino resin as described in the parent applications.

Toyopearl 650 M amino library resin was swollen in DMF; DMF/MeOH; 100% MeOH and 20% MeOH, then washed and equilibrated in citrate buffer (20 mM citrate, 140 mM NaCl, pH 7.4). 100 μl aliquots of resin were placed in columns.

7 ml tubes of whole blood were collected with heparin, EDTA, 20 mM citrate, CPD (commercial citrate anticoagulant) or no anticoagulant for serum preparation. The blood was split and 1 ml of each was treated with 10 μM PPACK, an irreversible thrombin inhibitor, (EMD Biosciences, San Diego, Calif.) for 5 min, mixing by rotation. Non-anticoagulated blood was allowed to clot at room temperature for 30 minutes and centrifuged to recover the serum. 1 ml of each sample was loaded onto 100 μl resin columns and allowed to flow by gravity. The unbound fraction was collected and the resins were washed with 2 ml citrate buffer, then the resins were transferred to a new tube containing 1 ml citrate buffer.

20 μl of each resin was mixed with 20 μl 2× LDS sample buffer (Invitrogen, Carlsbad, Calif.) and heated at 90° C. for 8 min. 20 μl of the supernatant and 20 μl 2×LDS sample buffer were combined and heated at 90° C. for 5 min. 20 μl was loaded in each lane of a 4-12% Bis-Tris gel run in MOPS buffer. The gel was run at 200V for 50 min, stained with SimplyBlue stain for 2-4 h; and destained with dH₂O for 4-6 hours.

Results:

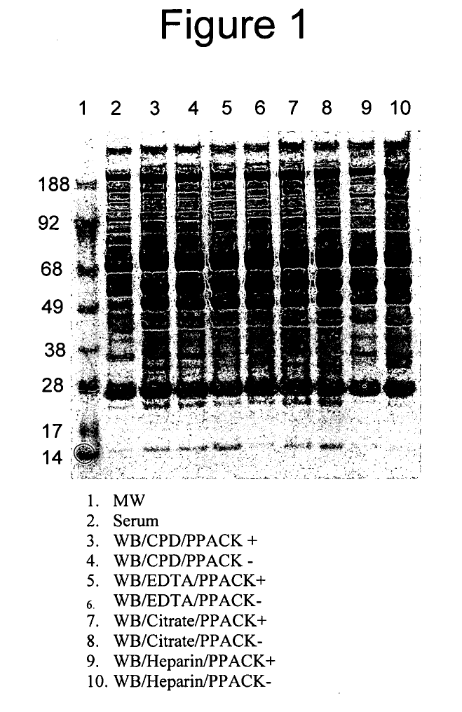

The results are presented in FIG. 1. There appear to be no differences between samples in a single anticoagulant with or without PPACK with the exception of the heparin samples; therefore, adding protease inhibitor to prevent clotting did not alter the binding and elution of the proteins. The citrate and EDTA anticoagulated samples are very similar to each other and differ from serum in the intensity of bands between 65 and 40 kD; the difference is more in intensity than in the presence or absence of a bands. There are more bands present in the lower molecular weight portion of the gel with the whole blood compared with the serum. Heparin-anticoagulated samples differ from the serum and the other anticoagulants with and without PPACK.

Thus, these data demonstrate that this invention can be used with whole blood, immediately upon collection, to bind proteins and analyze them without the need for preparation of either plasma or serum.

### Example 3

### Blood Versus Plasma

The ability to process samples directly from whole blood as opposed to from plasma will help to decrease artifacts that can be generated by plasma preparation, as well as increasing the stability of blood proteins and decreasing sample preparation time.

Methods:

Combinatorial peptide libraries of hexamer ligands were synthesized on Toyopearl AF amino650 M resin as described in the parent applications.

Resins were swollen and equilibrated in citrate buffer. 100 μl aliquots of library were transferred to columns. 1 ml of either whole blood or diluted plasma (1:1 in citrate buffer) was applied to each column and allowed to flow through by gravity. The unbound fraction was collected as flow through. Plasma was prepared from the whole blood flow through to compare the unbound fractions. The protein-loaded libraries were washed with 1 ml citrate buffer and bound proteins were eluted by heating at aliquot in 2× LDS ample buffer. 20 μl of 1:50 diluted untreated whole blood, plasma, or flow through samples, and 20 μl of 1:1 diluted eluates were loaded in lanes of a 4-12% Bis-Tris gel and run in MOPS buffer at 200 V. The proteins on the gel were visualized with SimplyBlue Safestain (Invitrogen).

Results:

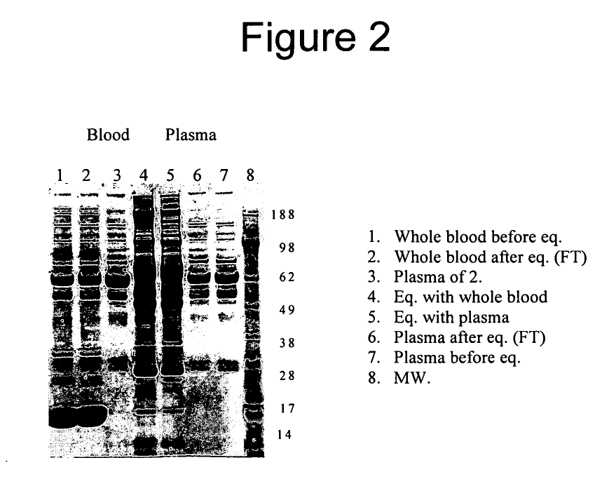

The pattern of proteins enriched from whole blood versus plasma (lanes 4 and 5, respectively, in FIG. 2) are very similar, indicating that the majority of proteins enriched are the same. Differences between the two (particularly in the high molecular weight range) may be due to the effect of producing plasma from the whole blood prior to processing. No clotting of blood was observed, or blockage from red blood cells, white cells or platelets was observed, nor hemolysis of red cells in the blood. These data indicate that the combinatorial peptide libraries may be used to process whole blood for sample preparation for proteomic analyis.

### Example 4

### Binding of a Specific Protein from Whole Blood

Previous results have demonstrated that plasma proteins can be bound by the library directly from whole blood. Further demonstration of the feasibility of using libraries of ligands to process whole blood is exemplified by concentration of a specific protein spiked into blood onto an affinity ligand synthesized on the same backbone on which the libraries are produced. This experiment was designed to evaluate the ability of two affinity ligands to bind infectious prion protein (PrPres) spiked into whole blood.

Methods:

Brain homogenate from scrapie-infected hamsters was diluted to 10% in sarkosyl and spiked into leukoreduced whole blood to a final concentration of 0.1% or into citrate buffer (20 mM citrate, 140 mM NaCl, pH 7.2). One aliquot of the homogenate was incubated for 24 hours at 37° C. 40 ml of incubated or not incubated, spiked blood or spiked buffer was applied to 0.5 ml column of two different trimer affinity resins with a contact time of one minute. Following washing, the resin was split: one aliquot was incubated with proteinase K (which degrades normal PrPc, leaving only PrPres present in the sample) and the remaining aliquot was untreated. All samples were incubated in 2× LDS sample buffer at 70° C. for 10 minutes, and the eluted material loaded on a 4-12% LDS-PAGE Bis-Tris gel run in MOPS buffer at 200V. Proteins in the gel were transferred to PVDF membranes and PrPres was detected with mouse anti-hamster PrP monoclonal antibody.

Results:

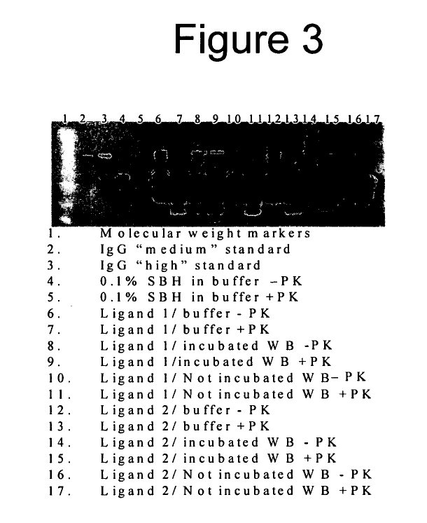

Results are presented in FIG. 3. All lanes containing proteins eluted from a resin illustrate the characteristic shift in molecular weight indicative of PrPres binding to the resins. Equivalent amounts of PrPres are detected in the elutes from all of the resins, regardless of the pre-incubation of the whole blood with the spike. There was no hemolysis of the red blood cells (RBCs), indicating that both the ligands and the backbone resin are compatible for use with whole blood. Furthermore, the RBCs did not block the column or appear to bind to the resin, nor did proteins in blood interfere with binding of PrPres to the affinity resins.

The combinatorial library was synthesized on hydroxypolymethacrylate beads obtained from Tosoh BioScience. The beads have a 5-15 atom linker between the resin and the ligand plus a spacer. The custom designed library was synthesized on Toyopearl 650 M AF Epoxy or AF amino resin (Tosoh Biosciences, Montgomeryville, Pa.) by Peptides International (Louisville, Ky.) based on Buettner, J. A., et al (Chemically derived peptide libraries: a new resin and methodology for lead identification. International Journal of Peptide & Protein Research 47, 70-83 (1996)). The libraries included 2′-naphthylalanine, and (except for glycine) had D-isomers at the amino terminal. The remaining amino acids were in the L-conformation. The library lacked cysteine and methionine throughout, and glutamine at the amino terminal. The epoxy resin library was synthesized using a cysteine spacer coupled through the sulfhydryl group and the amino resin had an alanine spacer.

### Example 5

### Use of Combinatorial Libraries (6-mers) for Improved Troponin Detection by Western Blotting

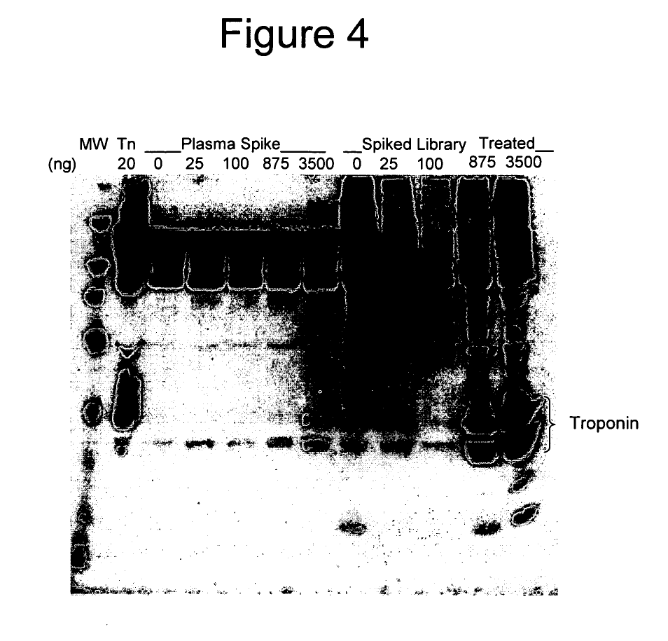

Troponin (Biodesign, Cat #A86862H Lot # 3C07903) was spiked into an aliquot of human whole blood at the following concentration: 0, 25, 100, 874 and 3,500 ng/ml. Blood was fractionated by centrifugation and the plasma collected from one part of the blood spiked samples (Plasma Spike). The second part of the blood samples (Spiked Treated) was incubated with 100 μl of the Toyopearl 650M library (see Example 1). Before incubation with blood, the Toyopearl 650M library was swollen in DMF, washed with 20% methanol (MeOH) and stored in 20% MeOH. Immediately before the experiment the Toyopearl 650M library was washed with PBS and equilibrated with citrate buffer. After incubation with blood samples, the library was washed three times with 1 ml of PBS to remove non-bound proteins. Bound proteins were eluted with 255 μl of 0.05M HCl and immediately neutralized with 85 μl 0.5M NaH₂PO₄ pH 7.5. Protein concentrations were evaluated in all samples, and an equal amount of protein (33 μg) from the eluates obtained from library, i.e. spiked blood and plasma samples, were loaded per lane of the gel for Western Blot analysis. Western Blot was performed according to standard procedure. Proteins were transferred onto PVDF membranes, the membranes blocked for non-specific binding and then stained with primary anti-Troponin I antibody (Biodesign, cat #H86207). Following incubation with primary antibody and washing, secondary goat anti-mouse IgG antibody labeled with peroxidase was added, further incubated and washed to remove non-bound antibody. WesternBreeze™ Chemiluminescent Detection Kit: (Invitrogen) was used for chemiluminescent detection of peroxidase. The results are shown in FIG. 4 and demonstrate that library-treatment significantly improves troponin detection in plasma samples. Moreover, the differential in troponin concentration between the different samples is maintained during library binding and elution.

### Example 6

### Use of Combinatorial Library (6-mers) for Improved Troponin Detection and Analysis by ELISA

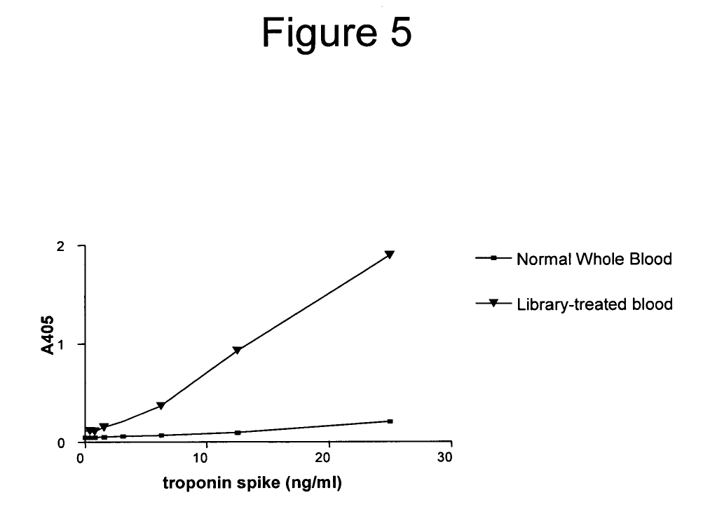

Troponin (Biodesign, Cat #A86862H Lot # 3C07903) was spiked into human plasma or human citrated blood at different concentrations (0-100 ng/ml). Toyopearl AF Amino 650 M library (see Example 1) was swollen in DMF, washed with, and stored in 20% MeOH. The library was washed with PBS and equilibrated with citrate buffer directly before the experiments were performed. Blood samples (1 ml) were added to the column containing resins (100 μl of bed volume). The resin was washed three times with 1 ml of PBS to remove non-bound proteins. To remove extra PBS, columns were centrifuged at 4000 g for 1 min in an Eppendorf tube. Bound proteins were eluted with 250 μl of 0.05M HCl and immediately neutralized with 85 μl 0.5M NaH₂PO₄ pH 7.5. Fifty μl of 1% BSA with 0.05% Tween 20 was added to stabilize the proteins. Evaluation of troponin in plasma and library treated blood samples was performed by Troponin I ELISA, Bio-Quant kit (#BQ 015C) according to the manufacturer's manual. The result is shown in FIG. 5. These data demonstrate that resin concentrates troponin from blood samples (V) spiked with troponin, improving the detection compared with untreated whole blood (O). The library maintains the concentration differential between samples, with more troponin detected from samples with higher initial concentrations than from samples with lower initial concentrations.

### Example 7

### Use of the Combinatorial Library (6-mers) for Improved Troponin Detection by ELISA

Troponin (Biodesign, Cat #A86862H Lot # 3C07903) was spiked into human plasma (plasma spiked samples, ⋄) or human citrated blood either before (blood pre-spiked samples, Δ) or after (blood post-spiked samples □) incubation with library. The range of troponin concentration was from 0 to 100 ng/ml. (Prior to incubation with samples, the Toyopearl AF Amino 650 M library was swollen in DMF, washed with and stored in 20% MeOH. The library resin was washed with PBS and equilibrated with citrate buffer directly before the experiments were performed.) One ml of blood samples spiked (pre-spiked samples, Δ), or non-spiked with troponin were applied to a column containing library resin (100 μl of bed volume). The resin was washed three times with 1 ml of PBS to remove non-bound proteins. To remove extra PBS, columns were centrifuged for 4,000 g for 1 min in an Eppendorf tube. Bound proteins were eluted with 250 μl of 0.05M HCl and immediately neutralized with 85 μl 0.5M NaH₂PO₄ pH 7.5. Eluates obtained from non-spiked blood samples were then spiked with 0-100 ng/ml of troponin (post-spiked samples, □).

Results:

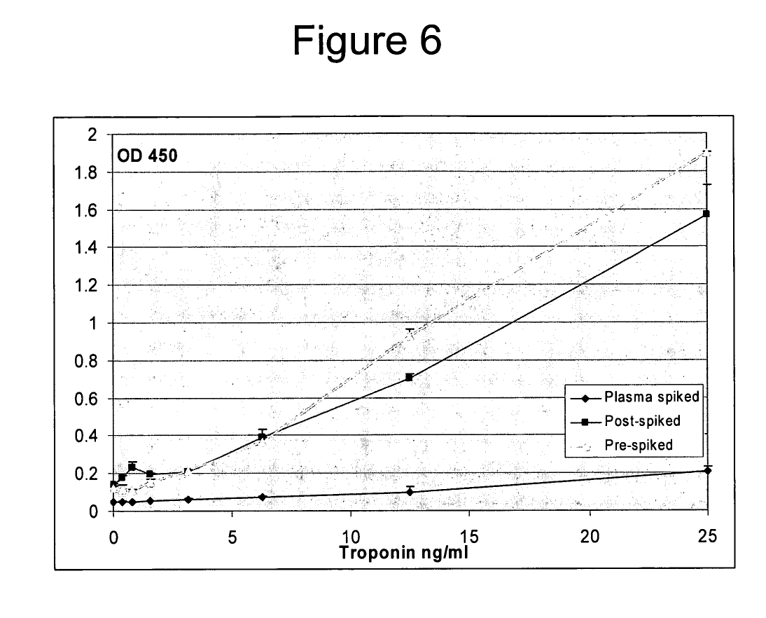

Evaluation of troponin in plasma and resin treated blood samples was performed by Troponin I ELISA, Bio-Quant kit (#BQ 015C) according to the manufacturer's manual. The results are presented in FIG. 6 and indicate that treatment of whole blood with lbrray significantly increases the sensitivity of troponin detection. These data also demonstrate that samples with the highest initial concentration of troponin elicit the highest signal in the assay, demonstrating that the differences in initial concentration are retained following processing with the library.

These data also demonstrate that plasma proteins recovered from the library and then spiked with troponin (□) produces a more sensitive response than troponin spiked into whole plasma (compare □ and ⋄). This is most likely due to the decrease in plasma proteins that interfere with the antibodies used in the ELISA by treatment with the library.

### Example 8

A Library of Hexamer Peptide Ligands was Synthesized on Toyopearl 650 M amino library (Tosoh Biosciences, Montgomeryville, Pa.) by Peptides International, Louisville, Ky. as described in the parent applications. The library was subsequently swollen and equilibrated in CPD (citrate, phosphate dextrose solution, Baxter Healthcare, Deerfield, Ill.) diluted 1:7 in phosphate buffered saline, pH 7.4 (140 mM NaCl). 500 μl aliquots of swollen, equilibrated library were dispensed into 10 ml Polyprep chromatography columns (Bio-Rad, Hercules, Calif.). Human CRP (Novagen, San Diego, Calif.) was spiked into 5 ml citrated whole blood to a final concentration of 100 ng/ml. The spiked blood was incubated with the equilibrated library for 1 hour at room temperature with rotation. Plasma proteins, including CRP, will bind to their corresponding ligands through affinity interactions.

After incubation, the unbound fraction was drained by gravity and the column was washed with 5 ml diluted CPD plus 0.05% Tween-20 (Sigma-Aldrich, St Louis, Mo.), followed by 2×5 ml diluted CPD. This produced the washed, “loaded” library. Bead blots were prepared as described in the parent application (U.S. patent application Ser. No. 10/414,523) by adding 10 μl of the blood loaded libraries containing approximately 25,000 beads, along with 2-3 μl of alignment beads, to 100 μl 0.5% low melting point agarose. Each mixture was poured on top of a 10 ml, of a 1.0% agarose gel (Pierce).

Alignment beads are used to improve identification and selection of CRP binding beads. Protein G sepharose beads are non-covalently bound with mouse IgG. This is detected by subsequent incubation with alkaline-phosphatase-labeled goat anti-mouse IgG (Pierce Biotechnology, Rockford, Ill.). The alignment beads generate a signal by forming a red precipitate on the beads upon incubation with chromogenic alkaline phosphatase substrate Fast-Red (Sigma-Aldrich, St. Louis, Mo.).

The gel was placed on a wick extending into a tank of transfer buffer. A PVDF membrane was placed on top of the gel, facing the beads, so that the bound proteins were transferred overnight by capillary action with transfer buffer and captured on the membrane. During transfer, the transfer buffer permeates through the gel and the membrane and in the process dissociates bound protein from the beads according to the strength of the affinity interaction and the composition of the transfer buffer. A variety of transfer conditions and transfer buffers may be used. To transfer the protein from a high affinity ligand, a strong chaotrope, such as 6M guanidine, is employed.

Upon removal of the membrane from the gel, the location of beads that had bound either mouse IgG from alignment beads or human CRP from loaded library was determined by detecting the presence of CRP using mouse anti-human CRP antibody (Sigma-Aldrich, St Louis, Mo.), followed by alkaline phosphatase labeled goat anti-mouse IgG secondary antibody (Pierce Biotechnology, Rockford, Ill.). This produced a film with spots indicating the position of detected protein. The film and the gel were superimposed and the spots aligned with beads. The majority of the spots aligned with red alignment beads. White beads associated with spots indicated potential CRP ligands. These beads were selected, and their ability to bind CRP was confirmed by re-equilibrating and re-incubating the beads with CRP in blood.

The experiments were repeated twice. Two beads from the spiked blood were selected. Ligand sequencing was performed by automated Edman degradation using a Procise 494 protein sequencer (Applied Biosystems, Inc, Foster City, Calif.).

The sequences derived from the beads from the spiked whole blood are: Leu-Gly-Thr-Tyr-Ile-Ala (SEQ ID NO: 1) and Gly-Asn-Gln-Lys-Trp-Gly (SEQ ID NO: 2), respectively where Nal represents 2′ naphthylalanine.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A method of detecting, identifying and characterizing the amount of a blood plasma-derived target that binds to a ligand, which method comprises:
(i) providing one thousand or more ligands, wherein each ligand is attached to a support to form one thousand or more ligand-support complexes
(ii) contacting the ligand-support complexes with a whole blood sample under conditions that allow at least one target to bind to at least one ligand-support complex, thereby forming one or more target-ligand support complexes
(iii) removing the non-bound fraction
(iv) eluting at least a portion of the blood plasma-derived target of at least two target-ligand-support complexes in an amount dependent on its concentration in the starting sample
(v) detecting the target whereupon the relative amount of the target that binds to one or more ligands is characterized

2. The method of claim 1, wherein in step (i), ten thousand, one hundred thousand or one million or more ligands are provided

3. The method of claim 1, wherein the blood is from a host afflicted with a disease.

4. The method of claim 1, wherein the targets are selected from the group consisting of cells, bacteria, viruses, yeast, microparticles, proteins, peptides, amino acids, nucleic acids, carbohydrates, lipids, drugs, synthetic inorganic compounds, synthetic organic compounds, isoforms of any of the foregoing, and combinations of any of the foregoing.

5. The method of claim 1, wherein the targets are tissue leakage proteins.

6. The method of claim 5, wherein the target is troponin.

7. The method of claim 5, wherein the target is a virus.

8. The method of claim 1, wherein the ligands are organic molecules.

9. The method of claim 8, wherein the organic molecules are selected from the group consisting of amino acids, peptides nucleic acids, carbohydrates, sugars, lipids, steroids, drugs, vitamins, and cofactors.

10. The method of claim 9, wherein the peptides consist essentially of about 1-15 amino acids.

11. The method of claim 1, wherein the support is a resin bead.

12. The method of claim 11, wherein the resin bead comprises a material selected from the group consisting of agarose, ethylene glycol, fluoropolymers, dimethacrylate, glycidol methacrylate, ethylene glycol dimethacrylate, pentaerythritol dimethacrylate, polyacrylate, polyesters, polyethylene glycol, polyhydroxymethacrylate, dextran, cellulose, polypropylene, polyethylene oxides, polysaccharide derivatives, and combinations of the foregoing.

13. The method of claim 12, wherein the resin is a polymer of glycidol methacrylate, polyethylene oxide, penta erythritol and ethylene glycol dimethacrylate, or analogs and combinations thereof.

14. The method of claim 13, wherein the resin is Toyopearl AF-Amino 650M resin.

15. The method of claim 1, wherein step (iv) is carried out in a medium containing a competitive binding agent, which binds to the target of at least one target-ligand-support complex, thereby causing the ligand to dissociate from at least a portion of the target.

16. The method of claim 1, wherein step (v) comprises performing mass spectrometry

17. The method of claim 1, wherein step (v) comprises performing gel-electrophoresis

18. The method of claim 1, wherein step (v) comprises performing an enzyme assay.

19. The method of claim 1, wherein step (v) comprises performing an immunological assay.

20. The method of claim 19, wherein the immunological assay is selected from the group consisting of an ELISA, nephelometry and Western blot based assay.

21. The method of claim 1, wherein step (v) comprises contacting cells with the eluted sample obtained in step (iv) and detecting a cellular response.

22. The method of claim 21, wherein the cellular response is cell death, growth or differentiation.

23. A method for detecting diagnostic biomarkers in blood comprising
(i) providing a first blood sample having a first phenotype and a first plurality of different targets
(ii) providing a second blood sample with a second phenotype and a second plurality of different targets
(iii) treating separately the first and the second plurality of different targets according to the method of claim 1, thereby creating a third and a fourth set of biosamples
(iv) identifying at least one target that is differentially present in the third and fourth set of biosamples, whereby the at least one target and its approximate concentration is a biomarker for distinguishing the first phenotype from the second phenotype.

24. The method of claim 1, that optionally comprises sub-pooling target-support-ligands prior to eluting one or more target-ligand-support complexes

25. The method of claim 24, wherein the target-ligand-support complexes are sub-pooled by a semi-permeable membrane.

26. The method of claim 25, wherein the targets are sub-pooled by equilibrium affinity dialysis using target specific affinity resins in one compartment and one thousand or more ligand-support-complexes in another.

27. The method of claim 24, wherein the one or more ligand-support complexes form target-ligand-support-complexes with the highly interactive targets.

28. The method of claim 27, wherein the highly interactive targets present in the sample are selected from the group consisting of fibrinogen, HDL, and LDL

29. The method of claim 24, wherein the target ligand support complexes are sub-pooled by physical separating of the resin beads by magnetic field, sedimentation rate, density or size.


SEQUENCE LISTING (2 sequences)

SEQ ID NO: 1 - protein, 6 aa, Artificial Sequence
  Synthetic
LGTYIA

SEQ ID NO: 2 - protein, 6 aa, Artificial Sequence
  Synthetic
GNQKWG